(19) United States
(12) Patent Application Publication
(10) Pub. No.: US 2014/0073993 A1
(43) Pub. Date: Mar. 13, 2014

(54) SYSTEMS AND METHODS FOR USING ISOLATED VOWEL SOUNDS FOR ASSESSMENT OF MILD TRAUMATIC BRAIN INJURY

(71) Applicant: University of Notre Dame du Lac, Notre Dame, IN (US)

(72) Inventors: Christian Poellabauer, Granger, IN (US); Patrick Flynn, Granger, IN (US); Nikhil Yadav, South Bend, IN (US)

(73) Assignee: University of Notre Dame du Lac, Notre Dame, IN (US)

(21) Appl. No.: 13/954,572

(22) Filed: Jul. 30, 2013

Related U.S. Application Data
(60) Provisional application No. 61/742,087, filed on Aug. 2, 2012.
(60) Provisional application No. 61/852,430, filed on Mar. 15, 2013.

(51) Int. Cl. A61B 5/00; A61B 7/04

(52) U.S. Cl. CPC A61B 5/4088; A61B 7/04; A61B 5/4064; A61B 5/7246; A61B 5/7282; A61B 5/7203; A61B 5/7267; A61B 5/0022; A61B 5/742; A61B 5/7475
USPC 600/586

(57) ABSTRACT

A system and method of identifying an impaired brain functionality such as a mild traumatic brain injury using speech analysis. In one example, recordings are taken on a device from athletes participating in a boxing tournament following each match. In one instance, vowel sounds are isolated from the recordings and acoustic features are extracted and used to train several one-class machine learning algorithms in order to predict whether an athlete is concussed.

### CROSS REFERENCE TO RELATED APPLICATION

This application is a non-provisional application claiming priority from U.S. Provisional Application Ser. No. 61/742,087, filed Aug. 2, 2012, and from U.S. Provisional Application Ser. No. 61/852,430, filed Mar. 15, 2013, each of which is incorporated herein by reference in its entirety.

### GOVERNMENT LICENSE RIGHTS

This invention was made with government support under Grant No. CNS-1062743 awarded by the National Science Foundation. The government has certain rights in the invention.

### FIELD OF THE DISCLOSURE

The present description relates generally to the detection and/or assessment of impaired brain function such as mild traumatic brain injuries and more particularly to systems and methods for using isolated vowel sounds for the assessment of mild traumatic brain injury.

### BACKGROUND OF RELATED ART

A concussion is a type of traumatic brain injury, or “TBI”, caused by a bump, blow, or jolt to the head that can change the way a person's brain normally works. Concussions can also occur from a fall or a blow to the body that causes the head and brain to move quickly back and forth. As such, concussions are typically common in contact sports. Health care professionals may describe a concussion as a “mild” traumatic brain injury, or “mTBI”, because concussions are usually not life-threatening. Even so, the short-term and long-term effects of a concussion can be very serious.

A concussion is oftentimes a difficult injury to diagnose. X-rays and other simple imaging of the brain often cannot detect signs of a concussion. Concussions sometimes can cause small amounts of bleeding usually in multiple areas of the brain, but to detect this bleeding the brain must typically be subject to magnetic resonance imaging (“MRI”). Most health care professionals, however, do not order an MRI for a concussion patient unless they suspect they have a life-threatening condition, such as major bleeding in the brain or brain swelling. This is because MRIs are usually very expensive and difficult to perform.

Accordingly, to diagnose a concussion physicians generally rely on the symptoms that the concussed individual reports or other abnormal patient signs such as disorientation or memory problems. As is oftentimes the case, many of the most widely known symptoms of concussions, such as amnesia or loss of consciousness, are frequently lacking in concussed individuals. Still further, some of the common symptoms also occur normally in people without a concussion, thereby leading to misdiagnosis.

In 2008, there were approximately 44,000 emergency department visits for sports-related mTBI. Repeated concussions can cause an increased risk of long term health consequences such as dementia and Parkinson's disease. In the United States, mTBI accounts for an estimated 1.6-3.8 million sports injuries every year and nearly 300,000 concussions are being diagnosed among young athletes every year. Athletes in sports such as football, hockey, and boxing are at a particularly large risk, e.g., six out of ten NFL athletes have suffered concussions, according to a study conducted by the American Academy of Neurology in 2000.

Concussions are also very frequent among soldiers, and are often called the “signature wound” of the Iraq and Afghanistan wars. Recent insights that the neuropsychiatric symptoms and long term cognitive impacts of blast or concussive injury of U.S. military veterans are similar to the ones exposed by young amateur American football players have led to collaborative efforts between athletics and the military. For example, the United Service Organizations Inc. recently announced that it will partner with the NFL to address the significant challenges in effectively detecting and treating mTBI.

The importance of procedures to assess mTBI has become increasingly important as the consequences of undiagnosed mTBIs become well known. Tests which are easy to administer, accurate, and not prone to unfair manipulation are required to properly assess mTBI.

There have been several previous studies related to motor speech disorders and their effects on speech acoustics. In one example, a research group conducted a study of the speech characteristics of twenty individuals with closed head injuries. The main result of that study was that the closed head injury subjects were found to be significantly less intelligible than normal non-neurologically impaired individuals, and exhibited deficits in the prosodic, resonatory, articulatory, respiratory, and phonatory aspects of speech production. Another study discovered an increase in vowel formant frequencies as well as duration of vowel sounds in persons with spastic dysarthria resulting from brain injury. In yet another study, a variation of the Paced Auditory Serial Addition Task (“PASAT”) test, which increases the demand on the speech processing ability with each subtest, was used to detect the impact of TBI on both auditory and visual facilities of the test takers. Still further, another study illustrated that tests on speech processing speed were affected by post-acute mTBI on a group of rugby players. Recently, a further study used acoustic features of sustained vowels to classify Parkinson's disease with Support Vector Machines (“SVM”) and Random Forests (“RF”), and showed that SVM outperformed RF. Finally, studies have also been conducted on the accommodation phenomenon, where test takers tend to adapt or adjust to unfamiliar speech patterns over time. Research has shown that accommodation is fairly rapid for healthy adults, and it has been studied as a speed based phenomenon.

While the above referenced references and studies generally work for their intended purposes, there is an identifiable need in the art of diagnosis (e.g., classification, detection, assessment, etc.) of mild traumatic brain injury as described herein.

### BRIEF DESCRIPTION OF THE DRAWINGS

For a better understanding of the present disclosure, reference may be had to various examples shown in the attached drawings.

FIG. 1 illustrates in block diagram form components of an example computer network environment suitable for implementing the example methods and systems disclosed.

FIG. 2 illustrates an example process diagram for implementing the example classification of mild traumatic brain injury disclosed.

FIG. 3 illustrates an example process diagram for implementing the example sound collection process.

FIG. 4 is a diagram showing an example extraction of a sample vowel sound.

FIG. 5 is a graph showing an example of performance measurements of the examples disclosed.

FIG. 6 is a graph showing example recall measurements in aggregate vowel sounds.

FIG. 7 is a graph showing example precision measurements in aggregate vowel sounds.

FIG. 8 is a graph showing example accuracy measurements in aggregate vowel sounds.

### DETAILED DESCRIPTION

The following description of example methods and apparatus is not intended to limit the scope of the description to the precise form or forms detailed herein. Instead the following description is intended to be illustrative so that others may follow its teachings.

The presently disclosed system and methods generally relate to the use of speech analysis for detection and assessment of mTBI. In the present examples disclosed herein, vowel sounds are isolated from speech recordings and the best acoustic features, which are most successful at assessing concussions are identified. Specifically, the present disclosure is concerned with the effects of concussion on specific speech features like formant frequencies, pitch, jitter, shimmer, and the like. Once analyzed, the present systems and methods use the relationship between TBI and speech to develop and provide scientifically based, novel concussion assessment techniques.

In one example use of the present disclosure, recordings were taken on a mobile device from athletes participating in a boxing tournament following each match. Vowel sounds were isolated from the recordings and acoustic features were extracted and used to train several one-class machine learning algorithms in order to predict whether the athlete was concussed. Prediction results were verified against the diagnoses made by a ringside medical team at the time of recording and performance evaluations showed prediction accuracies of up to 98%.

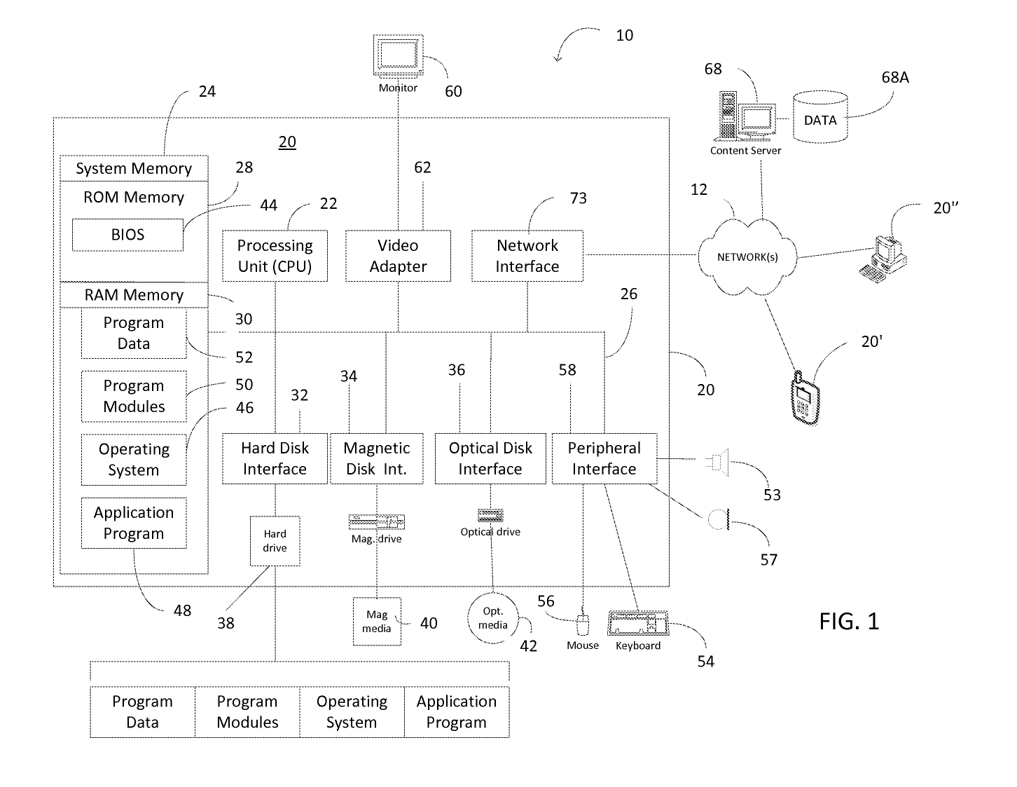

With reference to the figures, and more particularly, with reference to FIG. 1, the following discloses an example system 10 as well as other example systems and methods for providing detection (e.g. classification, assessment, diagnosis, etc.) of mild traumatic brain injury on a networked and/or standalone computer, such as a personal computer, tablet, or mobile device. To this end, a processing device 20″, illustrated in the exemplary form of a mobile communication device, a processing device 20′, illustrated in the exemplary form of a computer system, and a processing device 20 illustrated in schematic form, are provided with executable instructions to, for example, provide a means for a user, e.g., a healthcare provider, patient, technician, etc., to access a host system server 68 and, among other things, be connected to a hosted location, e.g., a website, mobile application, central application, data repository, etc.

Generally, the computer executable instructions reside in program modules which may include routines, programs, objects, components, data structures, etc. that perform particular tasks or implement particular abstract data types. Accordingly, those of ordinary skill in the art will appreciate that the processing devices 20, 20′, 20″ illustrated in FIG. 1 may be embodied in any device having the ability to execute instructions such as, by way of example, a personal computer, a mainframe computer, a personal-digital assistant (“PDA”), a cellular telephone, a mobile device, a tablet, an ereader, or the like. Furthermore, while described and illustrated in the context of a single processing device 20, 20′, 20″ those of ordinary skill in the art will also appreciate that the various tasks described hereinafter may be practiced in a distributed environment having multiple processing devices linked via a local or wide-area network whereby the executable instructions may be associated with and/or executed by one or more of multiple processing devices.

For performing the various tasks in accordance with the executable instructions, the example processing device 20 includes a processing unit 22 and a system memory 24 which may be linked via a bus 26. Without limitation, the bus 26 may be a memory bus, a peripheral bus, and/or a local bus using any of a variety of bus architectures. As needed for any particular purpose, the system memory 24 may include read only memory (ROM) 28 and/or random access memory (RAM) 30. Additional memory devices may also be made accessible to the processing device 20 by means of, for example, a hard disk drive interface 32, a magnetic disk drive interface 34, and/or an optical disk drive interface 36. As will be understood, these devices, which would be linked to the system bus 26, respectively allow for reading from and writing to a hard disk 38, reading from or writing to a removable magnetic disk 40, and for reading from or writing to a removable optical disk 42, such as a CD/DVD ROM or other optical media. The drive interfaces and their associated computer-readable media allow for the nonvolatile storage of computer-readable instructions, data structures, program modules, and other data for the processing device 20. Those of ordinary skill in the art will further appreciate that other types of non-transitory computer-readable media that can store data and/or instructions may be used for this same purpose. Examples of such media devices include, but are not limited to, magnetic cassettes, flash memory cards, digital videodisks, Bernoulli cartridges, random access memories, nano-drives, memory sticks, cloud based storage devices, and other read/write and/or read-only memories.

A number of program modules may be stored in one or more of the memory/media devices. For example, a basic input/output system (BIOS) 44, containing the basic routines that help to transfer information between elements within the processing device 20, such as during start-up, may be stored in ROM 28. Similarly, the RAM 30, hard drive 38, and/or peripheral memory devices may be used to store computer executable instructions comprising an operating system 46, one or more applications programs 48 (such as a Web browser, mobile application, etc.), other program modules 50, and/or program data 52. Still further, computer-executable instructions may be downloaded to one or more of the computing devices as needed, for example via a network connection.

To allow a user to enter commands and information into the processing device 20, input devices such as a keyboard 54, a pointing device 56 are provided. In addition, allow a user to enter and/or record sounds into the processing device 20, the input device may be a microphone 57 or other suitable device. Still further, while not illustrated, other input devices may include a joystick, a game pad, a scanner, a camera, touchpad, touch screen, motion sensor, etc. These and other input devices would typically be connected to the processing unit 22 by means of an interface 58 which, in turn, would be coupled to the bus 26. Input devices may be connected to the processor 22 using interfaces such as, for example, a parallel port, game port, firewire, a universal serial bus (USB), etc. To view information from the processing device 20, a monitor 60 or other type of display device may also be connected to the bus 26 via an interface, such as a video adapter 62. In addition to the monitor 60, the processing device 20 may also include other peripheral output devices, such as, for example, speakers 53, cameras, printers, or other suitable device.

As noted, the processing device 20 may also utilize logical connections to one or more remote processing devices, such as the host system server 68 having associated data repository 68A. The example data repository 68A may include any suitable healthcare data including, for example, patient information, collected data, physician records, manuals, etc. In this example, the data repository 68A includes a repository of at least one of specific or general patient data related to oratory information. For instance, the repository may include speech recordings from patients (e.g., athletes) and an aggregation of such recordings as desired.

In this regard, while the host system server 68 has been illustrated in the exemplary form of a computer, it will be appreciated that the host system server 68 may, like processing device 20, be any type of device having processing capabilities. Again, it will be appreciated that the host system server 68 need not be implemented as a single device but may be implemented in a manner such that the tasks performed by the host system server 68 are distributed amongst a plurality of processing devices/databases located at different geographical locations and linked through a communication network. Additionally, the host system server 68 may have logical connections to other third party systems via a network 12, such as, for example, the Internet, LAN, MAN, WAN, cellular network, cloud network, enterprise network, virtual private network, wired and/or wireless network, or other suitable network, and via such connections, will be associated with data repositories that are associated with such other third party systems. Such third party systems may include, without limitation, third party healthcare providers, additional data repositories, etc.

For performing tasks as needed, the host system server 68 may include many or all of the elements described above relative to the processing device 20. In addition, the host system server 68 would generally include executable instructions for, among other things, initiating a data collection process, an analysis regarding the detection and/or assessment of a traumatic brain injury, suggested protocol regarding treatment, etc.

Communications between the processing device 20 and the host system server 68 may be exchanged via a further processing device, such as a network router (not shown), that is responsible for network routing. Communications with the network router may be performed via a network interface component 73. Thus, within such a networked environment, e.g., the Internet, World Wide Web, LAN, cloud, or other like type of wired or wireless network, it will be appreciated that program modules depicted relative to the processing device 20, or portions thereof, may be stored in the non-transitory memory storage device(s) of the host system server 68.

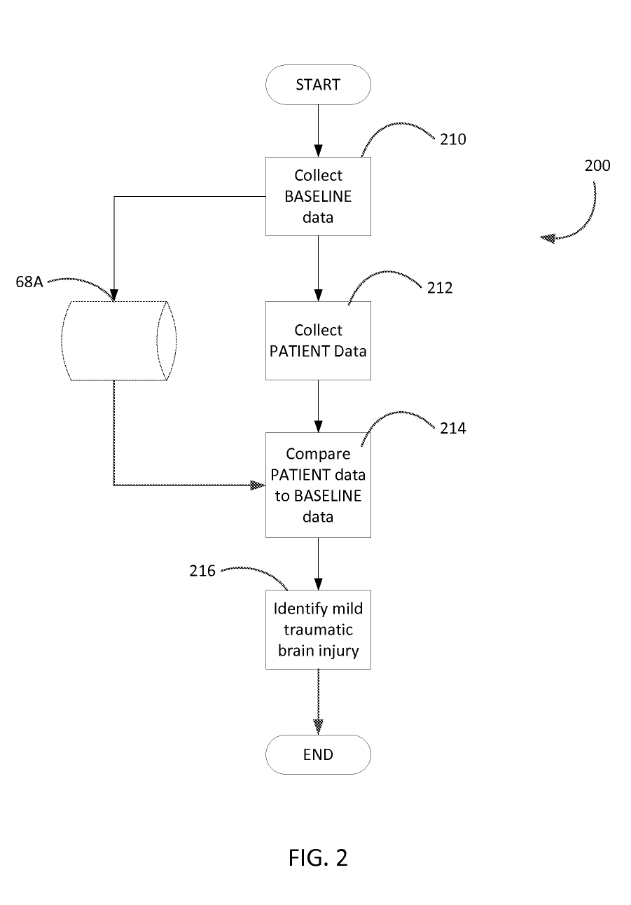

Turning now to FIG. 2, there is illustrated an example process 200 for detection and assessment of a mild traumatic brain injury. In the example process 200, baseline data is first collected at a block 210 and stored in the data repository 68. As will be described in detail herein, the collection process may include specific data gathering and processing, such as for example, the isolation of particular vowel sounds. It will be appreciated by one of ordinary skill in the art that while the examples described herein are generally noted as being patient specific, e.g., are directed to a baseline tied to a particular patient, the collection of baseline data may additionally or alternatively be directed to the aggregation of general, non-patient specific data such as, for example, generalized population data. For instance, in one example, there may be several recordings of at least one individual utilized to build a model of what a “healthy” or normalized voice should look like and compare a patient's voice to that model. In other examples, the patient's voice may simply be compared to an earlier recording from the same patient.

Once the baseline data has been collected, the process 200 may be utilized to specifically diagnose a mild traumatic brain injury at a block 212 by collecting patient data. In particular, when an mTBI is suspected, the example device 20 may be utilized to collect specific speech sequences from the patient utilizing any suitable equipment and any suitable speech pattern/sequence as desired. For instance, the collection of patient data may require the patient to read and/or recite a specific speech sequence, such as the same and/or similar sequence utilized in the collection of the baseline data at block 210. Similar to the baseline data, the collected diagnostic data may undergo the same example processing such as the isolation of the same particular vowel sounds.

After collection and processing of the patient's speech sequence, the system 200 may compare the collected patient data to the baseline data stored in the data repository at a block 214. For example, the process 214 may compare specific vowel and/or whole work sounds directly to determine differences in speech patterns between the baseline and the collect speech data. The comparison data may then be processed in a assessment algorithm at a block 216 to determine whether a mild traumatic brain injury has occurred and the assessment of the injury. As will be appreciated by one of ordinary skill in the art, the assessment process at block 216 may be singular, i.e., the identification of a mild traumatic brain injury via a single event, or may be based upon a feedback system wherein the process 200 “learns” through iterative trials and/or feedback data from independent sources, e.g., other diagnostic tests, to increase the accuracy of the assessment algorithm. In other words, the assessment step may entail the comparison of various speech markers (e.g., vowel sounds, full words, etc.) against an ever changing and evolving set of pre-determined thresholds in speech change to arrive at the ultimate diagnosis.

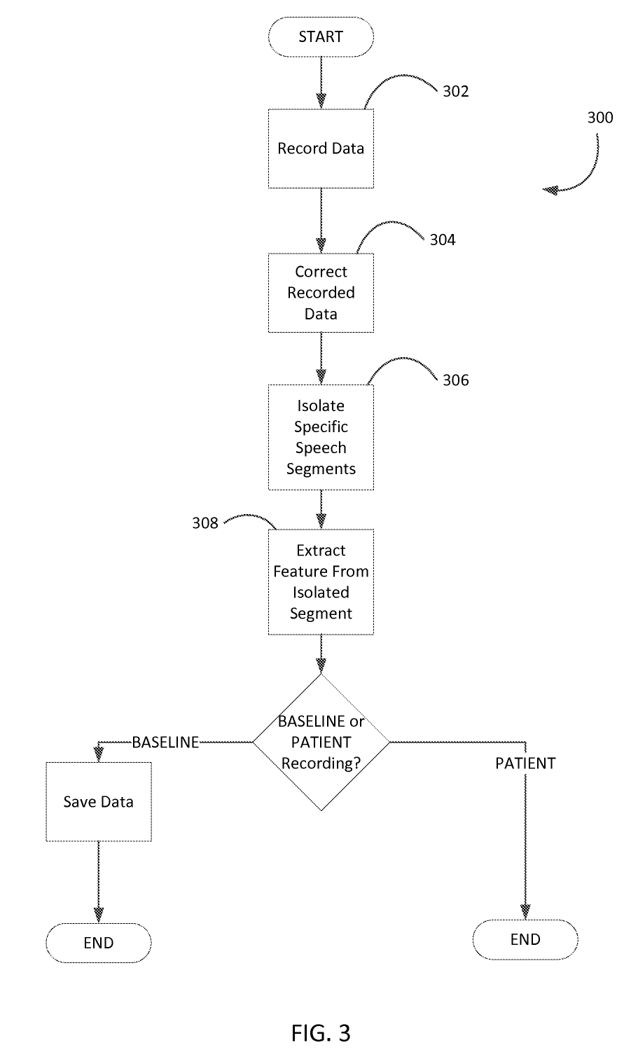

Referring now to FIG. 3, a more specific example of a process 300 of collecting baseline and/or patient data is described. In the example process 300, speech data is recorded utilizing the example device 20 and more particularly the microphone 57. In the instance where the data is baseline data, the recordings are performed prior to any activity, while in the instance where suspect mTBI data is being secured, the recordings take place during and/or after the suspect activity.

Once the speech data is recorded, the process 300 may optionally correct the recorded data at a block 304. In particular, the process 300 may perform noise correction and/or other suitable sound data processing as desired and/or needed. For instance, as is typical with any sound recording, some obtained recordings may include background noise and/or sound contamination, and therefore, the recordings may be processed for noise reduction, etc.

After any suitable recording processing, the example process 300 isolates a particular sound segment of interest, such as, for example, isolation of particular vowel segments at a block 306. For instance, in order to isolate the desired sound segment, the process 300 may first identify the onset of the desired sound-bite utilizing any suitable onset detection method as is well known to one of ordinary skill in the art. Once the onset of the desired sound is adequately determined, the recording may extend through a suitable length of time to record the sound.

Upon isolation of the particular segment of interest, the process 300 extracts features from the segment at a block 308. It will be appreciated by one of ordinary skill in the art that any of a number of features may be extracted from the segment. For instance, the speech features may include at least one of pitch, formant frequencies F₁-F₄, jitter, shimmer, mel-frequency cepstral coefficients (MFCC), or harmonics-to-noise ratio (HNR).

After the process 300 extracts the features at the block 308, the process 310 may determine whether the recording is a baseline recording or a diagnostic recording at a block 310. If the recording is a baseline recording, the data is stored at a block 312, individually and/or as a conglomerate in the data repository 68 as previously described. Alternatively, if the recording is a collection of patient data, the process 300 terminates with processing passing to the block 214 for diagnosis and/or assessment purposes.

With the process being sufficiently described, one example implementation of the disclosed systems and methods will be described in greater detail. For instance, in the identified example, speech recordings were acquired for a plurality of athletes before participation in several matches of a boxing tournament. The data was saved in the data repository and was utilized for both personal baseline and aggregate baseline processing. In this example, the subjects were recorded speaking a fixed sequence of digits that appeared on screen every 1.5 seconds for 30 seconds. The subjects spoke digit words in the following sequence: “two”, “five”, “eight”, “three”, “nine”, “four”, “six”, “seven”, “four”, “six”, “seven”, “two”, “one”, “five”, “three”, “nine”, “eight”, “five”, “one”, “two”, although it will be understood that various other sounds and/or sequences may be utilized as desired.

Each subject was recorded on a mobile tablet by a directional microphone and as noted, several of the recordings contained background noise or background speakers. Speech was sampled at 44.1 kHz with 16 bits per sample in two channels and later mixed down to mono-channel for analysis.

For purposes of demonstration of the baseline and post-activity differences, in the identified trial example, the obtained recordings were split into training/test data and grouped into three classes: baseline (training), post-healthy (test), and post-mTBI (test). Table 1 below summarizes these classes and gives the number of recordings in each class. A few speakers have recordings in both the post-healthy class and the post-mTBI class if they were diagnosed with mTBI in a match following acquisition of the post-healthy recordings. In such cases, the recordings were taken in separate matches of the tournament. Thus, the number of test recordings is greater than the number of training recordings but both sets of data are mutually exclusive.

TABLE 1
Classes of speech recording

| | Number of | |
|---|---|---|
| Class of Speech | Recordings | Description |
| Baseline | 105 | Recorded prior to tournament; all |
| | | subjects healthy. |
| Post-Activity (healthy) | 101 | Recorded following preliminary match; |
| | | subjects not independently diagnosed |
| | | with mTBI and assumed healthy. |
| Post-Activity (mTBI) | 7 | Recorded at subject's final match of |
| | | participation; subjects independently |
| | | diagnosed with mTBI. |

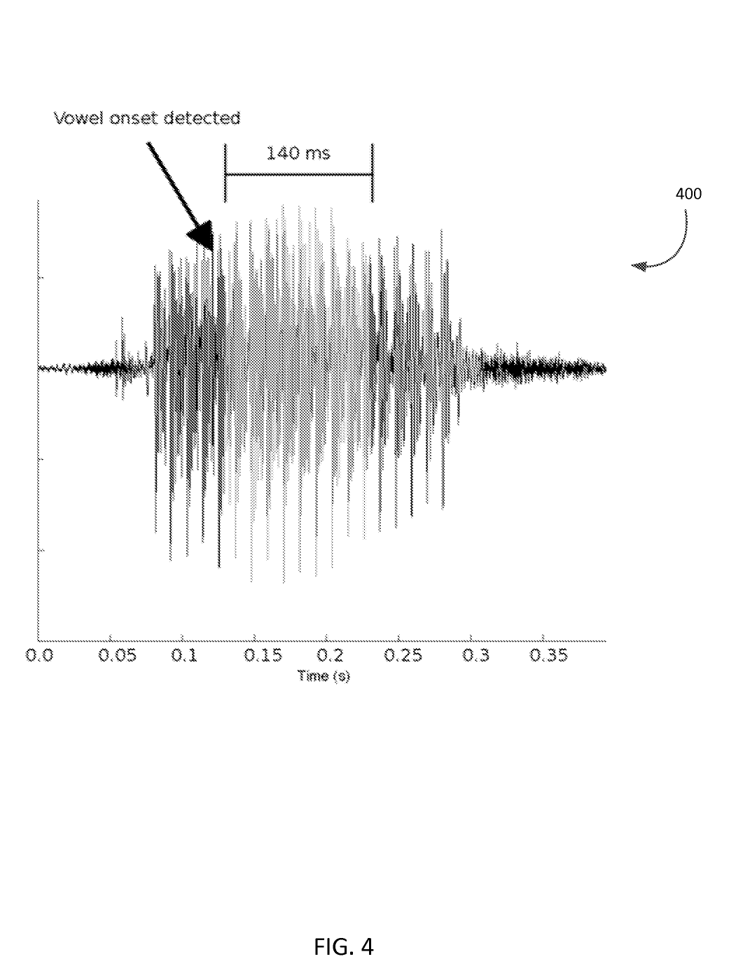

Vowel segments were then isolated from each speech recording by first locating vowel onsets and then extracting 140 ms of speech for each vowel sound, following each onset. In this example, onsets were detected using an adaptation of a well known method for onset detection in isolated words. For example, FIG. 4 illustrates a graphical illustration 400 of an example of the isolation process, where a vowel onset 402 was detected, and the /ai/ vowel sound was isolated from the recording of a subject speaking the phrase “five.” Repeating this process yielded a total of 3786 vowel sounds among each of the three classes of recordings. In particular, Table 2 shows the number of segments isolated from each class of recordings. It will be appreciated that each class contains a different number of vowel sounds. This is because the number of whole recordings differs for each class and occasionally vowel onsets are missed during the isolation process.

TABLE 2
Number of vowel sound instances isolated
from each class of speech recordings.

| Sound | Baseline | Post-Healthy | Post-mTBI |
|---|---|---|---|
| /i/-three | 150 | 160 | 10 |
| /I/-six | 190 | 188 | 12 |
| /e/-eight | 162 | 160 | 10 |
| /ε/-seven | 207 | 200 | 14 |
| /Λ/-one | 205 | 189 | 13 |
| /u/-two | 212 | 224 | 18 |
| /o/-four | 204 | 202 | 14 |
| /ai/-five | 313 | 302 | 21 |
| /ai/-nine | 205 | 190 | 11 |

Eight speech features were investigated in this example: pitch, formant frequencies F₁-F₄, jitter, shimmer, and harmonics-to-noise ratio (HNR). While jitter and shimmer are typically measured over long sustained vowel sounds, the use of jitter over short-term time intervals may also be used in analyzing pathological speech. For purposes of this example, pitch was estimated using autocorrelation and formants were estimated via a suitable transform, such as a fast Fourier transform (FFT).

Jitter is a measure of the average variation in pitch between consecutive cycles, and is given by the equation:

$$Jitter = \frac{{\sum }_{i = 2}^{N} \vert {T}_{i} - {T}_{i - 1} \vert}{N - 1}$$

where N is the total number of pitch periods and Tᵢ is the duration of the i^{th} pitch period.

Shimmer, meanwhile, is a measure of the average variation in amplitude between consecutive cycles, given by the equation:

$$Shimmer = \frac{{\sum }_{i = 2}^{N} \vert {A}_{i} - {A}_{i - 1} \vert}{N - 1}$$

where N is the total number of pitch periods and A, is the amplitude of the i^{th} pitch period.

Once the features where extracted, various combinations of extracted features were selected as inputs to several one-class Support Vector Machines (SVM) classifiers. In this example, SVMs are supervised learning models with associated learning algorithms that analyze data and recognize patterns, used for assessment and regression analysis. The basic SVM takes a set of input data and predicts, for each given input, which of two possible classes forms the output, making it a non-probabilistic binary linear classifier. In one example, a LIBSVM (e.g., a library of support vector machines) implementation was used. In this particular example, a one-class classifier was chosen because the baseline data did not include any mTBI speech and the number of recordings in the post-mTBI class was significantly lower than the number of recordings in post-healthy. Features were scaled to the ranges 0-1 by dividing each feature by the maximum value of that feature in the training set. In order to find the optimal combination of features for each vowel sound, each possible combination of at least three features was used to train and test the classifier for each vowel sound.

In order to classify the individual vowel sounds, an individual classifier was trained for each vowel sound in the baseline class. In this instance, the /ai/ sound in the word “five” was treated separately from the /ai/ sound in “nine” because the consonantal context differs between these words, i.e., the /ai/ sound in “five” occurs between two fricatives while the /ai/ sound in “nine” occurs between two nasal consonants. Each sound in the post-healthy and post-mTBI classes was tested and the prediction results were used to compute three standard performance measures: recall, precision, and accuracy. In particular, recall gives the percentage of correctly predicted mTBI segments and was defined as:

$$Recall = \frac{\# of segments correctly classised mTBI}{Total \# of tue mTBI segments}$$

Precision, meanwhile, was defined as the rate at which the mTBI predictions were correct, and was defined as:

$$Precision = \frac{\# of segments correctly classified mTBI}{Total \# of segments classified mTBI}$$

Finally, accuracy was considered the percentage of segments that were classified correctly (either mTBI or healthy), and was defined as:

$$Accuracy = \frac{\# correctly classified segments}{Total \# of segments}$$

The classifier achieved accuracies approaching 70% for some feature combinations and recall rates as high as 92% for other combinations. Table 3 shows the features that achieved maximum accuracy for each vowel sound. In any case where equal accuracies were achieved for more than one feature combination, the combination yielding the best recall is listed.

TABLE 3
Vowel sounds and features achieving maximum accuracy.

| Vowel | Recall | Prec. | Acc. | Features* |
|---|---|---|---|---|
| /i/ | 0.4 ( 4/10) | 0.069 | 0.65 | F₃, F₄, J, H, P |
| /I/ | 0.5( 6/12) | 0.11 | 0.71 | F₁, F₄, S, H |
| /e/ | 0.6( 6/10) | 0.083 | 0.59 | F₄, J, H |
| /E/ | 0.5( 7/14) | 0.089 | 0.63 | F₃, S, H, P |
| /2/ | 0.54( 7/13) | 0.095 | 0.64 | F₄, S, H, P |
| /u/ | 0.61( 11/18) | 0.11 | 0.59 | F₃, F₄, J |
| /o/ | 0.79( 11/14) | 0.14 | 0.67 | F₁, F₄, S |
| /ai/five | 0.76( 16/21) | 0.13 | 0.66 | F₁, F₃, J, S, H, P |
| /ai/nine | 0.64( 7/11) | 0.097 | 0.66 | F₂, F₃, F₄ |

Where Fₙ = frequency of formant n, J = jitter, S = shimmer, H = harmonics-to-noise ratio, P = pitch frequency.

Still further, Table 4 shows the feature combinations that achieved maximum recall for each vowel sound. In any case where an equal recall was achieved for more than one combination of features, the combination yielding the best accuracy is shown. In any case where multiple feature combinations yielded equal maximum recalls and equal accuracies, the combination with the fewest number of features was chosen. In the case of the /e/ sound, two combinations yielded recalls of 80% and accuracies of 56%. In this case, all features from both combinations were used despite a reduction in accuracy for that sound by 3%.

TABLE 4
Vowel sounds and features achieving maximum recall.

| Vowel | Recall | Prec. | Acc. | Features* |
|---|---|---|---|---|
| /i/ | 0.9( 9/10) | 0.11 | 0.55 | F₁, F₃, S |
| /I/ | 0.92( 11/12) | 0.1 | 0.51 | F₁, F₂, P |
| /e/ | 0.8( 8/10) | 0.093 | 0.53 | F₂, F₄, S, P |
| /E/ | 0.79( 11/14) | 0.11 | 0.57 | F₂, J, S |
| /2/ | 0.77( 10/13) | 0.1 | 0.55 | F₁, F₄, P |
| /u/ | 0.89( 16/18) | 0.13 | 0.55 | F₂, F₃, J, S, P |
| /o/ | 0.79( 11/14) | 0.14 | 0.67 | F₁, F₄, S |
| /ai/five | 0.81( 17/21) | 0.14 | 0.66 | F₁, F₂, F₃, J, S, H, P |
| /ai/nine | 0.82( 9/11) | 0.12 | 0.65 | F₁, F₂, F₃ |

Where Fₙ = frequency of formant n, J = jitter, S = shimmer, H = harmonics-to-noise ratio, P = pitch frequency.

Once the recorded data was obtained, the assessment of boxers' speech recordings by using each vowel was elaborated. Specifically, a tradeoff between accuracy and recall can be seen from Table 3 and Table 4 for most vowel sounds. In order to keep false negatives to a minimum, a higher importance was placed on recall of mTBI vowel sounds. Similarly to individual vowel sound segments, performance of whole recording assessment was evaluated by measuring recall, precision, and accuracy measures.

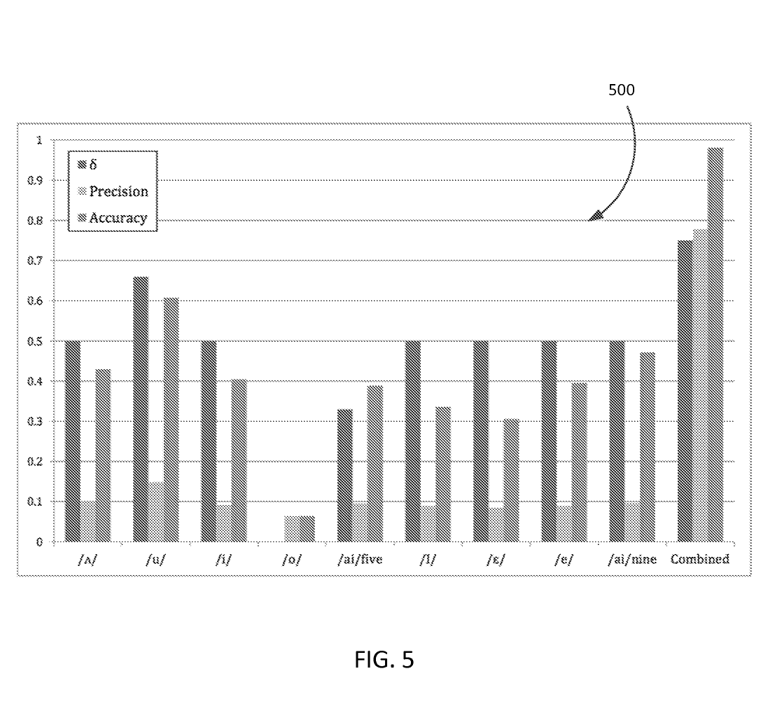

Using the feature combinations that achieved maximum recall for individual vowel sound segments (Table 4), individual one-class SVM classifiers were again trained for each vowel sound in the baseline class of recordings. Next, each speech recording in post-healthy and post-mTBI was classified as a whole by classifying each instance of a specific vowel sound from the recording. A threshold δ was defined, such that the speech recording was classified as mTBI speech if the following relationship held true:

$$δ \leq \frac{N ( v )}{M ( v )}$$

where N gives the number of instances of the vowel sound v classified as mTBI in the recording and M gives the total number of instances of the vowel sound v that could be isolated in the recording. Several trials were performed in which each recording was classified and performance was measured with the vowel sound v as a different vowel sound for each trial, i.e., each unique vowel sound corresponds to a single trial. For each trial, the threshold δ was adjusted until recall of mTBI recordings reached 100%. The corresponding value of the threshold δ is shown in FIG. 5, which illustrates performance measurements 500 for each assessment trial and the minimum threshold δ yielding 100% mTBI recall.

A final assessment trial was performed in which all vowel sounds were aggregated such that a recording was classified as mTBI speech if the following relationship held true:

$$δ \leq \frac{{\sum }_{v ∈ V} N ( v )}{{\sum }_{v ∈ V} M ( v )}$$

where V is the set of all vowel sounds isolated from that recording. Referring again to FIG. 5, there is illustrated a comparison of performance measurements and shows the minimum threshold δ for each trial that resulted in recall of all seven mTBI recordings, specifically, the “combined” trial in FIG. 5, shows the performance measures for the aggregate trial along with the corresponding threshold δ that achieved 100% recall of mTBI recordings.

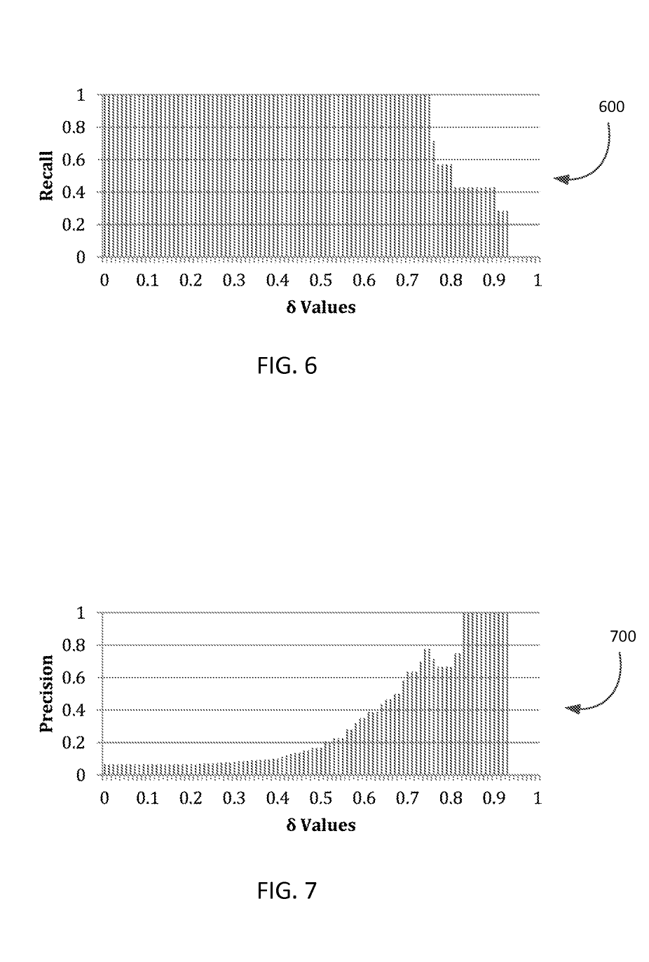

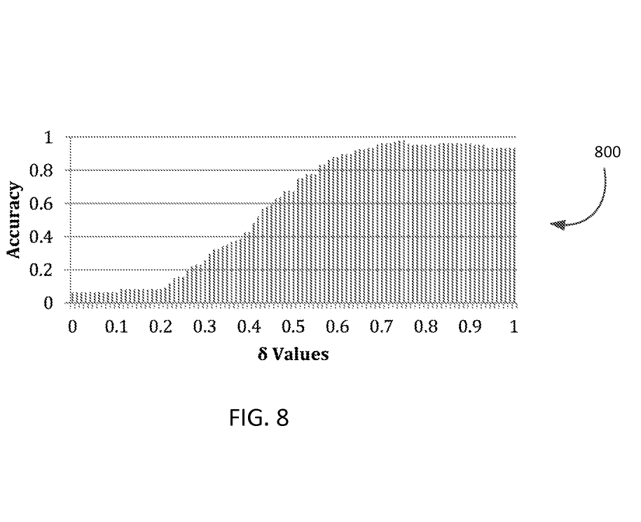

FIGS. 6-8 illustrate and example recall 600, precision 700, and accuracy 800 measurements, respectively, as the value of threshold δ was adjusted in the aggregate trial. It can be seen that as the threshold δ increases, recall 600 decreases while precision 700 and accuracy 800 tend to increase.

For the aggregate trial, the threshold δ=0.75 resulted in best accuracy while still recalling all mTBI recordings. A value of the threshold δ=0:75 means that when the assessment system encounters a speech recording in which more than 75% of all isolated vowel sound segments are classified mTBI, the entire recording is classified mTBI. This threshold δ was able to recall all seven mTBI recordings with an accuracy of 0.982 and precision of 0.778.

By using speech analysis on isolated vowel sounds extracted from any suitable application including a mobile application, the vowel acoustic features that give the best recall and accuracy measures in identifying concussed athletes are therefore identified. It will be appreciated by one of ordinary skill in the art that various combinations of vowel sounds and/or acoustic features may be selected with varying degrees of effective threshold δ values. Furthermore, different noise reduction techniques may be applied to the recordings to give samples that are ideal for extraction of the vowel sounds and features.

Still further, as will be understood by one of ordinary skill in the art, an implementation of vowel sounds analysis for concussion assessment in on-line mode (e.g., using an appropriate storage facility such as a cloud-based feed-back approach), or off-line (e.g. no network connect required) may be utilized. In both cases, a sideline physician (e.g., coach, trainer, etc.) at contact sports will get near real-time results to help identify suspected concussion cases.

Finally, while the present examples are direct to isolation of vowel sounds from recording a spoken fixed sequence of digits, the present disclosure may utilize monosyllabic and/or multisyllabic words rather than numbers as desired. In this example, the differing sounds may be utilized to emphasize words with the vowel sounds and their acoustic features identified as the most successful in assessing concussive behavior in one example of the present invention.

It will be appreciated by one of ordinary skill in the art that the example systems and methods described herein may be utilized on a networked and/or a non-networked (e.g., local) system as desired. For example, in at least one example, the server 68 may perform at least a portion of the speech analysis and the result sent to the device 20, while in yet other examples (e.g., offline, non-networked, etc.) the speech processing is performed directly on the device 20 and/or other suitable processor as needed. The non-networked and/or offline system may be utilized in any suitable situation, including the instance where a network is unavailable. In this case, the baseline and processing logic may be stored directly on the device 20.

Yet further, while the present examples are specifically directed to the detection and/or assessment of mild traumatic brain injury, it will be understood that the example systems and methods disclosed may be used for detecting other impaired brain functions such as Parkinson's disease, intoxication, stress, or the like.

Although certain example methods and apparatus have been described herein, the scope of coverage of this patent is not limited thereto. On the contrary, this patent covers all methods, apparatus, and articles of manufacture fairly falling within the scope of the appended claims either literally or under the doctrine of equivalents.

We claim:

1. A method of identifying a mild traumatic brain injury comprising:
using a sound recording device to capture spoken sound recording data from at least one individual at a first point in time to establish a spoken sound baseline;
storing the spoken sound baseline in a data repository;
capturing a spoken sound from a patient at a second point in time subsequent to the first point in time;
comparing the spoken sound to the spoken sound baseline retrieved from the data repository; and
using the comparison of the spoken sound to the spoken sound baseline retrieved from the data repository to determine if the patient has experienced a mild traumatic brain injury between the first point in time and second point in time.

2. A method as recited in claim 1, wherein the captured spoken sound recording data is from a single individual.

3. A method as recited in claim 2, wherein the patient is the single individual.

4. A method as recited in claim 1, wherein the spoken sound baseline is a normalization of captured spoken sound recordings from a plurality of individuals.

5. A method as recited in claim 1, further comprising removing unwanted noise from at least one of the recorded spoken sound baseline or the captured spoken sound.

6. A method as recited in claim 1, further comprising isolating a speech segment from at least one of the recorded spoken sound baseline or the captured spoken sound.

7. A method as recited in claim 6, wherein isolated speech segment is a vowel sound.

8. A method as recited in claim 6, wherein isolating the speech segment further comprises identifying the onset of the speech segment via an onset detection routine.

9. A method as recited in claim 1, further comprising identifying a speech feature in at least one of the recorded spoken sound baseline or the captured spoken sound.

10. A method as recited in claim 9, wherein the speech feature is at least one of pitch, formant frequencies F₁-F₄, jitter, shimmer, mel-frequency cepstral coefficients, or harmonics-to-noise ratio.

11. A method as recited in claim 1, wherein the comparison of the spoken sound to the spoken sound baseline comprises a learning model with an associated learning algorithm.

12. A method as recited in claim 11, wherein the learning model analyzes the comparison data and recognizes patterns for assessment and regression analysis.

13. A method as recited in claim 11, wherein comparison of the spoken sound to the spoken sound baseline is performed via a support vector machine.

14. A non-transient, computer-readable media having stored thereon instructions for assisting a healthcare provider in identifying a mild traumatic brain injury, the instructions comprising:
receiving from a sound recording device, spoken sound recording data from at least one individual at a first point in time to establish a spoken sound baseline;
storing the spoken sound baseline in a data repository;
receiving spoken sound from a patient at a second point in time subsequent to the first point in time;
comparing the spoken sound to the spoken sound baseline retrieved from the data repository; and
determining if the patient has experienced a mild traumatic brain injury between the first point in time and second point in time using the comparison of the spoken sound to the spoken sound baseline retrieved from the data repository.

15. A computer-readable media as recited in claim 14, wherein the captured spoken sound recording data is from a single individual.

16. A computer-readable media as recited in claim 15, wherein the patient is the single individual.

17. A computer-readable media as recited in claim 14, wherein the spoken sound baseline is a normalization of captured spoken sound recordings from a plurality of individuals.

18. A computer-readable media as recited in claim 1, further comprising isolating a speech segment from at least one of the recorded spoken sound baseline or the captured spoken sound.

19. A computer-readable media as recited in claim 18, wherein isolated speech segment is a vowel sound.

20. A computer-readable media as recited in claim 14, wherein comparison of the spoken sound to the spoken sound baseline is performed via a support vector machine.

21. A method of identifying an impaired brain function comprising:
using a sound recording device to capture spoken sound recording data from at least one individual at a first point in time to establish a spoken sound baseline;
storing the spoken sound baseline in a data repository;
capturing a spoken sound from a patient at a second point in time subsequent to the first point in time;
comparing the spoken sound to the spoken sound baseline retrieved from the data repository; and
using the comparison of the spoken sound to the spoken sound baseline retrieved from the data repository to determine if the patient has experienced an impaired brain function between the first point in time and second point in time.
